(19) United States
(12) Patent Application Publication
(10) Pub. No.: US 2021/0007592 A1
(43) Pub. Date: Jan. 14, 2021

(54) Tubing With Integrated Optical Fiber, Medical Devices, And Methods Thereof

(71) Applicant: Bard Access Systems, Inc., Salt Lake City, UT (US)

(72) Inventor: Chase Thompson, Bountiful, UT (US)

(21) Appl. No.: 16/926,414

(22) Filed: Jul. 10, 2020

Related U.S. Application Data
(60) Provisional application No. 62/873,794, filed on Jul. 12, 2019.

(51) Int. Cl. A61B 1/07; A61B 5/00; A61B 1/00

(52) U.S. Cl. CPC A61B 1/07; A61B 1/0011; A61B 1/00147; A61B 5/0084

(57) ABSTRACT

Integrated tubing is disclosed including a tubing wall, one or more lumens, and a longitudinal bead of the integrated tubing including one or more optical fibers disposed therein. Each lumen of the one or more lumens is defined at least in part by the tubing wall. The longitudinal bead of the integrated tubing is between opposing sides of the tubing wall. A medical device is also disclosed including a catheter tube and an integrated stylet. The catheter tube includes a catheter-tube wall and one or more lumens of the catheter tube. Each lumen of the one or more lumens is defined at least in part by the catheter-tube wall. The integrated stylet includes one or more optical fibers. The catheter tube also includes a longitudinal bead between opposing sides of the catheter tube having the one or more optical fibers disposed therein. Method for manufacturing the foregoing are also disclosed.

### PRIORITY

This application claims the benefit of priority to U.S. Provisional Application No. 62/873,794, filed Jul. 12, 2019, which is incorporated by reference in its entirety into this application.

### BACKGROUND

At times, a tip of the peripherally inserted central catheter (“PICC”) or central venous catheter (“CVC”) can move becoming displaced from an ideal position in a patient's superior vena cava (“SVC”). A clinician believing such a PICC or CVC has displaced typically checks for displacement by chest X-ray and replaces the PICC or CVC if necessary. However, X-rays expose patients to ionizing radiation. Therefore, there is a need for clinicians to easily and safely check for displacement of PICCs and CVCs for replacement thereof if necessary.

Disclosed herein is integrated tubing with integrated optical fiber, medical devices, and methods thereof that address the foregoing.

### SUMMARY

Disclosed herein is integrated tubing including, in some embodiments, a tubing wall, one or more lumens, and a longitudinal bead of the integrated tubing including one or more optical fibers disposed therein. Each lumen of the one or more lumens is defined at least in part by the tubing wall. The longitudinal bead of the integrated tubing is between opposing sides of the tubing wall.

In some embodiments, the integrated tubing has one lumen.

In some embodiments, the longitudinal bead is incorporated into the tubing wall.

In some embodiments, the integrated tubing has two or more lumens.

In some embodiments, each lumen of the two or more lumens has approximately a same volume as another lumen of the two or more lumens in an arbitrary length of the integrated tubing.

In some embodiments, at least one lumen of the two or more lumens has a different volume than another lumen of the two or more lumens in an arbitrary length of the integrated tubing.

In some embodiments, the integrated tubing further includes one or more septa of the integrated tubing. Each lumen of the two or more lumens is further defined at least in part by the one or more septa.

In some embodiments, the longitudinal bead is incorporated into the tubing wall.

In some embodiments, the longitudinal bead is incorporated into at least one septum of the one or more septa.

In some embodiments, the longitudinal bead is incorporated into each septum of the one or more septa.

In some embodiments, the longitudinal bead has one optical fiber disposed therein.

In some embodiments, the longitudinal bead has a bundle of optical fibers disposed therein.

In some embodiments, the integrated tubing is bump tubing.

Also disclosed herein is medical device including, in some embodiments, a catheter tube and an integrated stylet. The catheter tube includes a catheter-tube wall and one or more lumens of the catheter tube. Each lumen of the one or more lumens is defined at least in part by the catheter-tube wall. The integrated stylet includes one or more optical fibers. The catheter tube also includes a longitudinal bead between opposing sides of the catheter tube having the one or more optical fibers disposed therein.

In some embodiments, the catheter tube has one lumen.

In some embodiments, the longitudinal bead is incorporated into the catheter-tube wall.

In some embodiments, the catheter tube has two or more lumens.

In some embodiments, each lumen of the two or more lumens has approximately a same volume as another lumen of the two or more lumens in an arbitrary length of the catheter tube.

In some embodiments, at least one lumen of the two or more lumens has a different volume than another lumen of the two or more lumens in an arbitrary length of the catheter tube.

In some embodiments, the medical device further includes one or more septa of the catheter tube. Each lumen of the two or more lumens is further defined at least in part by the one or more septa.

In some embodiments, the longitudinal bead is incorporated into the catheter-tube wall.

In some embodiments, the longitudinal bead is incorporated into at least one septum of the one or more septa.

In some embodiments, the longitudinal bead is incorporated into each septum of the one or more septa.

In some embodiments, the longitudinal bead has one optical fiber disposed therein.

In some embodiments, the longitudinal bead has a bundle of optical fibers disposed therein.

In some embodiments, a distal-end portion of the catheter tube has a smaller diameter than a proximal-end portion of the catheter tube. The distal-end portion of the catheter tube configured to interface with a patient's anatomy. The proximal-end portion of the catheter tube is configured to interface with procedural equipment or instrumentation.

In some embodiments, the medical device is a PICC or a CVC.

Also disclosed herein is a method for manufacturing integrated tubing including, in some embodiments, threading at least one optical fiber through a die of an extruder; forcing a molten polymeric material through the die around the optical fiber to form the integrated tubing with the optical fiber disposed within a longitudinal bead of the integrated tubing; and cooling the integrated tubing in a cooling bath by pulling the integrated tubing through the cooling bath with a puller.

In some embodiments, the method further includes coiling the integrated tubing onto a spool of the integrated tubing with a coiler.

In some embodiments, a rate of pulling the integrated tubing with the puller is increased to create portions of the integrated tubing with a smaller diameter or decreased to create portions of the integrated tubing with a larger diameter.

In some embodiments, the method further includes cutting the integrated tubing into predetermined lengths of the integrated tubing with a cutter.

In some embodiments, the cutter is synchronized with the puller such that each predetermined length of the integrated tubing includes a first-end portion of the predetermined length having a smaller diameter than a second-end portion of the predetermined length.

In some embodiments, each predetermined length of the integrated tubing is a catheter tube. The first-end portion of the predetermined length of the integrated tubing is a distal-end portion of the catheter tube configured to interface with a patient's anatomy. The second-end portion of the predetermined length of the integrated tubing is a proximal-end portion of the catheter tube configured to interface with procedural equipment or instrumentation.

These and other features of the concepts provided herein will become more apparent to those of skill in the art in view of the accompanying drawings and following description, which describe particular embodiments of such concepts in greater detail.

### DRAWINGS

FIG. 1 illustrates a cross-section of single-lumen integrated tubing with integrated optical fiber in accordance with some embodiments.

FIG. 2 illustrates a cross-section of two-lumen integrated tubing with integrated optical fiber in accordance with some embodiments.

FIG. 3A illustrates a cross-section of three-lumen integrated tubing with integrated optical fiber in accordance with some embodiments.

FIG. 3B illustrates a cross-section of alternative three-lumen integrated tubing with integrated optical fiber in accordance with some embodiments.

FIG. 4A illustrates a CVC including a two-lumen catheter tube with integrated optical fiber in accordance with some embodiments.

FIG. 4B illustrates a detailed view of a proximal-end portion of a catheter tube of the CVC of FIG. 4A.

FIG. 5 illustrates a method of manufacturing integrated tubing with integrated optical fiber in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

At times, a tip of the PICC or CVC can move becoming displaced from an ideal position in a patient's SVC. A clinician believing such a PICC or CVC has displaced typically checks for displacement by chest X-ray and replaces the PICC or CVC if necessary. However, X-rays expose patients to ionizing radiation. Therefore, there is a need for clinicians to easily and safely check for displacement of PICCs and CVCs for replacement thereof if necessary.

Disclosed herein is integrated tubing with integrated optical fiber, medical devices, and methods thereof that address the foregoing.

### Integrated Tubing

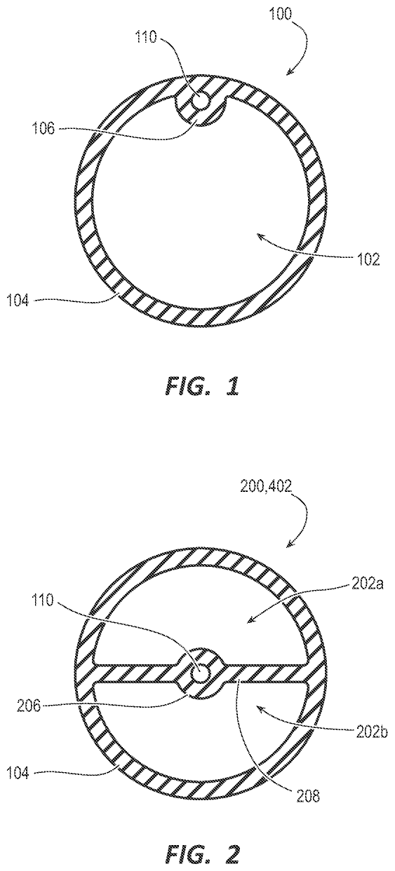

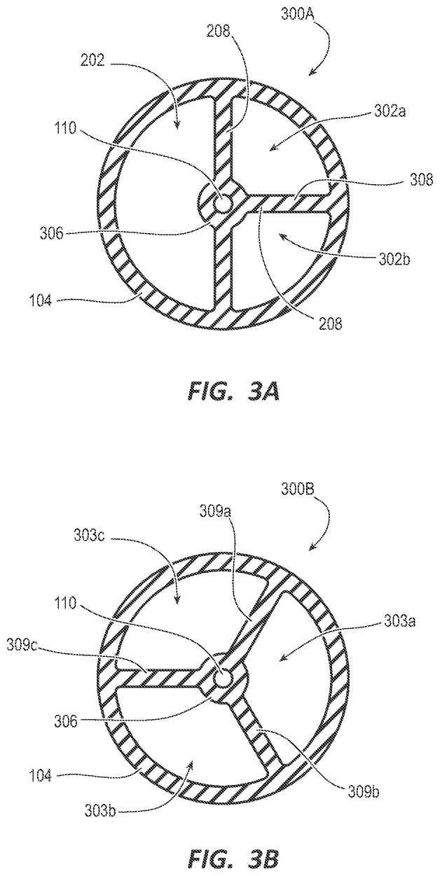

FIG. 1 illustrates a cross-section of single-lumen integrated tubing 100 with integrated optical fiber 110 in accordance with some embodiments. FIG. 2 illustrates a cross-section of two-lumen integrated tubing 200 with the integrated optical fiber 110 in accordance with some embodiments. FIG. 3A illustrates a cross-section of three-lumen integrated tubing 300A with the integrated optical fiber 110 in accordance with some embodiments. FIG. 3B illustrates a cross-section of alternative three-lumen integrated tubing 300B with the integrated optical fiber 110 in accordance with some embodiments.

As shown in FIG. 1, the integrated tubing 100 includes a tubing wall 104, a lumen 102, and a longitudinal bead 106 of the integrated tubing 100 including one or more optical fibers 110 disposed therein. Likewise, as shown in FIG. 2, the integrated tubing 200 includes a tubing wall 104, two lumens 202a and 202b, a longitudinal bead 206 of the integrated tubing 200 including the one or more optical fibers 110 disposed therein. As shown in FIG. 3A, the integrated tubing 300A includes a tubing wall 104, three lumens 202, 302a, and 302b, and a longitudinal bead 306 of the integrated tubing 300A including the one or more optical fibers 110 disposed therein. Likewise, as shown in FIG. 3B, the integrated tubing 300B includes a tubing wall 104, three lumens 303a, 303b, and 303c, and a longitudinal bead 306 of the integrated tubing 300B including the one or more optical fibers 110 disposed therein. As such, the integrated tubing disclosed herein includes at least a tubing wall, one or more lumens, and a longitudinal bead of the integrated tubing including one or more optical fibers disposed therein.

With respect to the integrated tubing 100 of FIG. 1, the lumen 102 is defined at least in part by the tubing wall 104, particularly an internal surface of the tubing wall 104.

The longitudinal bead 106 of the integrated tubing 100 is between opposing sides of the tubing wall 104. In other words, the longitudinal bead 106 is inside of the integrated tubing 100. Indeed, the longitudinal bead 106 is incorporated into the tubing wall 104, particularly the internal surface of the tubing wall 104. As such, the lumen 102 is further defined at least in part by the longitudinal bead 106.

With respect to the integrated tubing 200 of FIG. 2, each lumen of the two lumens 202a and 202b is defined at least in part by the tubing wall 104, particularly an internal surface of the tubing wall 104.

The integrated tubing 200 further includes a septum 208 of the integrated tubing 200. Each lumen of the two lumens 202a and 202b is further defined at least in part by the septum 208. As shown, the septum 208 separates the integrated tubing 200 into two halves such that each lumen of the two lumens 202a and 202b of the integrated tubing 200 has approximately a same volume as the other lumen of the two lumens 202a and 202b for an arbitrary length of the integrated tubing 200. However, the septum 208 need not separate the integrated tubing 200 into two equal halves in accordance with the foregoing. For example, instead of the septum 208 extending from 3 o'clock to 9 o'clock as it would if the cross-section of the integrated tubing 200 of FIG. 2 was a face of a clock, the septum 208 could extend from 2 o'clock to 10 o'clock. So configured, each lumen of the two lumens 202a and 202b of the integrated tubing 200 would have a different volume than the other lumen of the two lumens 202a and 202b for an arbitrary length of the integrated tubing 200.

The longitudinal bead 206 of the integrated tubing 200 is between opposing sides of the tubing wall 104. In other words, the longitudinal bead 206 is inside of the integrated tubing 200. Indeed, the longitudinal bead 206 is incorporated into the septum 208, particularly a medial portion of the septum 208 such that the longitudinal bead 206 is coaxial with a central axis of the integrated tubing 200. However, the longitudinal bead 206 can alternatively be incorporated into the septum 208 between the medial portion of the septum 208 and the tubing wall 104. That said, the longitudinal bead 206 need not be incorporated into the septum 208 at all. Instead, the longitudinal bead 206 can be incorporated into the tubing wall 104 like that of the integrated tubing 100. Regardless of how the longitudinal bead 206 is incorporated into the integrated tubing 200, at least one lumen of the two lumens 202a and 202b of the integrated tubing 200 is further defined at least in part by the longitudinal bead 206.

With respect to the integrated tubing 300A of FIG. 3A, each lumen of the three lumens 202, 302a, and 202b is defined at least in part by the tubing wall 104, particularly an internal surface of the tubing wall 104.

The integrated tubing 300A further includes two septa 208 and 308 of the integrated tubing 300A. Each lumen of the three lumens 202, 302a, and 302b is further defined at least in part by the septum 208. Each lumen of the two lumens 302a and 302b is even further defined at least in part by the septum 308. As shown, the septum 208 separates the integrated tubing 300B into a first set of two halves, and the septum 308 separates one half of the first set of two halves into a second set of two more halves. As a result, each lumen of the two lumens 302a and 302b of the integrated tubing 300A has approximately a same volume as the other lumen of the two lumens 302a and 302b for an arbitrary length of the integrated tubing 200, and the lumen 202 has at least double the volume of the two lumens 302a and 302b for the arbitrary length of the integrated tubing 200. However, the septum 208 need not separate the integrated tubing 200 into the first set of two halves and the septum 308 need not separate one half of the first set of two halves into a second set of two more halves in accordance with the foregoing. For example, instead of the septum 208 extending from 12 o'clock to 6 o'clock as it would if the cross-section of the integrated tubing 300A of FIG. 3A was a face of a clock, the septum 208 could extend from 11 o'clock to 7 o'clock. Likewise, instead of the septum 308 extending from 3 o'clock to a medial portion of the septum 208, the septum 308 could extend from 2 o'clock to the medial portion of the septum 208 or from 2 o'clock to between the medial portion of the septum 208 and the tubing wall 104. So configured, each lumen of the three lumens 202, 302a, and 302b of the integrated tubing 300A could have a different volume than another lumen of the three lumens 202, 302a, and 302b for an arbitrary length of the integrated tubing 300A.

The longitudinal bead 306 of the integrated tubing 300A is between opposing sides of the tubing wall 104. In other words, the longitudinal bead 306 is inside of the integrated tubing 300A. Indeed, the longitudinal bead 306 is incorporated into the septa 208 and 308, particularly a medial portion of the septum 208 and an end portion of the septum 308 such that the longitudinal bead 306 is coaxial with a central axis of the integrated tubing 300A. However, the longitudinal bead 306 can alternatively be incorporated into the septum 208 between the medial portion of the septum 208 and the tubing wall 104 or in a medial portion of the septum 308. That said, the longitudinal bead 306 need not be incorporated into either septum of the septa 208 and 308 at all. Instead, the longitudinal bead 306 can be incorporated into the tubing wall 104 like that of the integrated tubing 100. Regardless of how the longitudinal bead 306 is incorporated into the integrated tubing 300A, at least one lumen of the three lumens 202, 302a, and 302b of the integrated tubing 300A is further defined at least in part by the longitudinal bead 306.

With respect to the integrated tubing 300B of FIG. 3B, each lumen of the three lumens 303a, 303b, and 303c is defined at least in part by the tubing wall 104, particularly an internal surface of the tubing wall 104.

The integrated tubing 300B further includes three septa 309a, 309b, and 309c of the integrated tubing 300B. Each lumen of the three lumens 303a, 303b, and 300c is further defined at least in part by two septa of the three septa 309a, 309b, and 309c. As shown, the three septa 309a, 309b, and 309c separate the integrated tubing 300B into thirds such that each lumen of the three lumens 303a, 303b, and 300c of the integrated tubing 300B has approximately a same volume as another lumen of the three lumens 303a, 303b, and 300c for an arbitrary length of the integrated tubing 200. However, the three septa 309a, 309b, and 309c need not separate the integrated tubing 300B into three equal thirds in accordance with the foregoing. For examples, see the integrated tubing 300A of FIG. 3A and the related description set forth above. Configured in accordance with the foregoing examples, each lumen of the three lumens 303a, 303b, and 303c of the integrated tubing 300B could have a different volume than another lumen of the three lumens 303a, 303b, and 303c for an arbitrary length of the integrated tubing 300B.

The longitudinal bead 306 of the integrated tubing 300B is between opposing sides of the tubing wall 104. In other words, the longitudinal bead 306 is inside of the integrated tubing 300B. Indeed, the longitudinal bead 306 is incorporated into the septa 309a, 309b, and 309c, particularly an end portion of each septum of the septa 309a, 309b, and 309c such that the longitudinal bead 306 is coaxial with a central axis of the integrated tubing 300B. However, the longitudinal bead 306 can alternatively be incorporated into a medial portion of any septum of the septa 309a, 309b, and 309c. That said, the longitudinal bead 306 need not be incorporated into any septum of the septa 309a, 309b, and 309c at all. Instead, the longitudinal bead 306 can be incorporated into the tubing wall 104 like that of the integrated tubing 100. Regardless of how the longitudinal bead 306 is incorporated into the integrated tubing 300B, at least one lumen of the three lumens 203a, 303b, and 303C of the integrated tubing 300B is further defined at least in part by the longitudinal bead 306.

The integrated tubing 100, 200, 300A, or 300B can be integrated bump tubing having a diameter that varies along its length. When the integrated bump tubing is cut into predetermined lengths in accordance with the how the diameter of the integrated bump tubing varies along it length, each predetermined length of the integrated bump tubing can have a first-end portion having a smaller diameter than a second-end portion such that the first-end portion of the predetermined length of the integrated bump tubing is configured to interface with, for example, mammalian anatomy and the second-end portion of the predetermined length of the integrated bump tubing is configured to interface with medical equipment or instrumentation.

The integrated tubing 100, 200, 300A, or 300B including the tubing wall 104, the septa 208, 308, or 309a-c, and the longitudinal bead 106, 206, or 306 is either an inorganic polymer such as silicone or an organic polymer such as polyurethane.

The optical fiber 110 disposed in the longitudinal bead 106, 206, or 306 of the integrated tubing 100, 200, 300A, or 300B includes one optical fiber or a bundle of two or more optical fibers of silica glass or an organic polymer, optionally cladded with cladding of another silica glass or organic polymer having a lower refractive than that of the optical fiber, a buffer coating of yet another organic polymer, or a combination thereof. Each optical fiber of the foregoing optical fibers has a diameter between about 8 μm and about 25 μm.

### Medical Devices

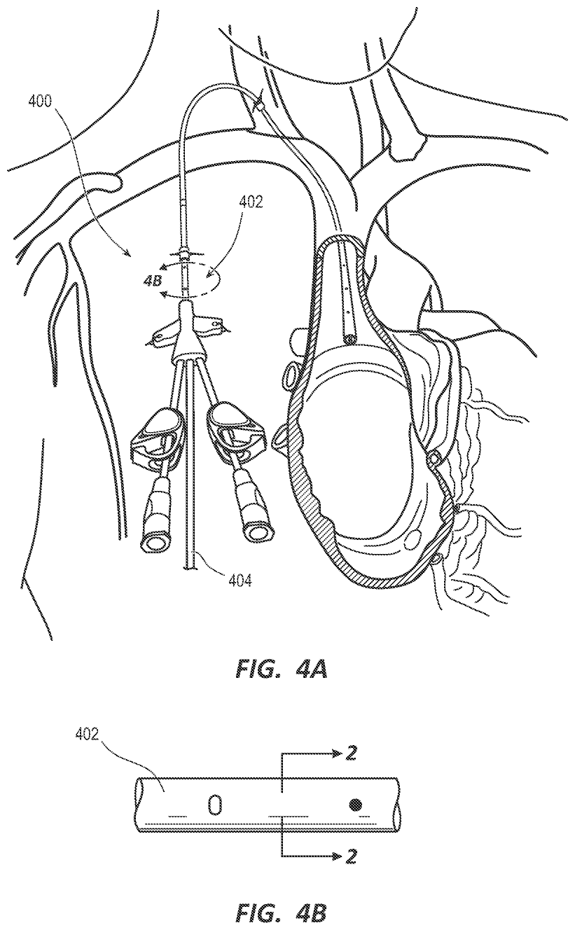

FIG. 4A illustrates a CVC 400 including a two-lumen catheter tube 402 with an integrated optical-fiber stylet 110 in accordance with some embodiments. FIG. 4B illustrates a detailed view of a proximal-end portion of the catheter tube 402 of the CVC 400 of FIG. 4A. Again, FIG. 2 illustrates the cross-section of the two-lumen integrated tubing 200 with the integrated optical fiber 110 in accordance with some embodiments; however, the catheter tube 402 is a predetermined length of the integrated tubing 200, so features of the integrated tubing 200 are incorporated into the catheter tube 402 as set forth below.

As shown in FIG. 4A, the CVC 400 includes the catheter tube 402, which, as set forth in more detail below, includes two catheter-tube lumens 202a and 202b, a bifurcated hub having two hub lumens correspondingly fluidly connected to the two catheter-tube lumens 202a and 202b, two extension legs, each extension leg having an extension-leg lumen fluidly connected to a hub lumen of the two hub lumens, and a Luer connector connected to each extension leg of the two extension legs. The CVC 400 optionally includes a stylet extension 404 extending from the hub of the CVC 400 for interfacing the integrated optical-fiber stylet 110 of the catheter tube 402 with procedural equipment or instrumentation. The stylet extension 404 can be a skived portion of the catheter tube 402 including the optical-fiber stylet 110 or the skived portion of the catheter tube 402 disposed in another tube (e.g., an extension tube).

With respect to the catheter tube 402 of FIGS. 2, 4A, and 4B, each lumen of the two catheter-tube lumens 202a and 202b is defined at least in part by a catheter-tube wall 104, particularly an internal surface of the catheter-tube wall 104.

The catheter tube 402 further includes a septum 208 of the catheter tube 402. Each lumen of the two catheter-tube lumens 202a and 202b is further defined at least in part by the septum 208. As shown, the septum 208 separates the catheter tube 402 into two halves such that each lumen of the two catheter-tube lumens 202a and 202b of the catheter tube 402 has approximately a same volume as the other lumen of the two catheter-tube lumens 202a and 202b for an arbitrary length of the catheter tube 402. However, the septum 208 need not separate the catheter tube 402 into two equal halves in accordance with the foregoing. For example, instead of the septum 208 extending from 3 o'clock to 9 o'clock as it would if the cross-section of the catheter tube 402 of FIG. 2 was a face of a clock, the septum 208 could extend from 2 o'clock to 10 o'clock. So configured, each lumen of the two catheter-tube lumens 202a and 202b of the catheter tube 402 would have a different volume than the other lumen of the two catheter-tube lumens 202a and 202b for an arbitrary length of the catheter tube 402.

A longitudinal bead 206 of the catheter tube 402 in which the integrated optical-fiber stylet 110 is disposed is between opposing sides of the catheter-tube wall 104. In other words, the longitudinal bead 206 is inside of the catheter tube 402. Indeed, the longitudinal bead 206 is incorporated into the septum 208, particularly a medial portion of the septum 208 such that the longitudinal bead 206 is coaxial with a central axis of the catheter tube 402. However, the longitudinal bead 206 can alternatively be incorporated into the septum 208 between the medial portion of the septum 208 and the catheter-tube wall 104. That said, the longitudinal bead 206 need not be incorporated into the septum 208 at all. Instead, the longitudinal bead 206 can be incorporated into the catheter-tube wall 104 like that of the integrated tubing 100. Regardless of how the longitudinal bead 206 is incorporated into the catheter tube 402, at least one lumen of the two catheter-tube lumens 202a and 202b of the catheter tube 402 is further defined at least in part by the longitudinal bead 206.

The catheter tube 402 is a predetermined length of the integrated tubing 200 cut such that a distal-end portion of the catheter tube 402 has a smaller diameter than a proximal-end portion of the catheter tube 402. The distal-end portion of the catheter tube 402 is configured to interface with a patient's anatomy. The proximal-end portion of the catheter tube 402 is configured to interface with procedural equipment or instrumentation.

Like the integrated tubing 100, 200, 300A, or 300B, the catheter tube 402 including the catheter-tube wall 104, the septum 208, and the longitudinal bead 206 is either an inorganic polymer such as silicone or an organic polymer such as polyurethane.

The integrated optical-fiber stylet 110 disposed in the longitudinal bead 206 of the catheter tube 402 includes one optical fiber or a bundle of two or more optical fibers of silica glass or an organic polymer, optionally cladded with cladding of another silica glass or organic polymer having a lower refractive than that of the optical fiber, a buffer coating of yet another organic polymer, or a combination thereof. Each optical fiber of the foregoing optical fibers has a diameter between about 8 μm and about 25 μm.

While the CVC 400 set forth above is a two-lumen CVC having the two-lumen catheter tube 402, other medical devices are possible such as a two-lumen PICC analogous to the CVC 400. In addition, one- or three-lumen CVCs or PICCs are possible, wherein each one-lumen CVC or PICC has a catheter tube and integrated optical-fiber stylet 110 corresponding to the integrated tubing 100, and wherein each three-lumen CVC or PICC has a catheter tube and integrated optical-fiber stylet 110 corresponding to the integrated tubing 300A or 300B.

### Manufacturing

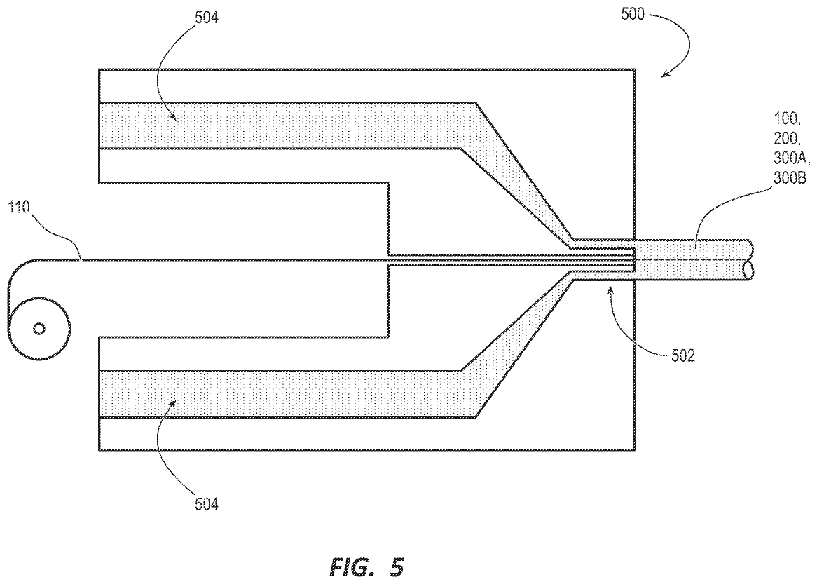

FIG. 5 illustrates a method of manufacturing the integrated tubing 100, 200, 300A, or 300B with the integrated optical fiber 110 in accordance with some embodiments.

A method for manufacturing the integrated tubing 100, 200, 300A, or 300B includes threading the optical fiber 110 (e.g., one-by-one or from a spool of the optical fiber) through a die 502 of an extruder 500; forcing a molten polymeric material 504 such as silicone or polyurethane through the die 502 around the optical fiber 110 to form the integrated tubing 100, 200, 300A, or 300B with the optical fiber 110 disposed within the longitudinal bead 106, 206, or 306 of the integrated tubing 100, 200, 300A, or 300B; and cooling the integrated tubing 100, 200, 300A, or 300B in a cooling bath by pulling the integrated tubing through the cooling bath with a puller. A rate of pulling the integrated tubing 100, 200, 300A, or 300B with the puller is increased to create portions of the integrated tubing 100, 200, 300A, or 300B with a smaller diameter or decreased to create portions of the integrated tubing 100, 200, 300A, or 300B with a larger diameter.

The method can further include coiling the integrated tubing 100, 200, 300A, or 300B onto a spool of the integrated tubing 100, 200, 300A, or 300B with a coiler.

The method can further include cutting the integrated tubing 100, 200, 300A, or 300B into predetermined lengths of the integrated tubing 100, 200, 300A, or 300B with a cutter. The cutter can be synchronized with the puller such that each predetermined length of the integrated tubing 100, 200, 300A, or 300B includes a first-end portion of the predetermined length having a smaller diameter than a second-end portion of the predetermined length. As set forth above, each predetermined length of the integrated tubing 100, 200, 300A, or 300B can be a catheter tube such as the catheter tube 402 formed of a predetermined length of the integrated tubing 200. The first-end portion of such a predetermined length of the integrated tubing 200 is a distal-end portion of the catheter tube 402 configured to interface with a patient's anatomy. The second-end portion of such a predetermined length of the integrated tubing 200 is a proximal-end portion of the catheter tube 402 configured to interface with procedural equipment or instrumentation.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. Integrated tubing, comprising:
a tubing wall of the integrated tubing;
one or more lumens of the integrated tubing, each lumen of the one or more lumens defined at least in part by the tubing wall; and
a longitudinal bead of the integrated tubing between opposing sides of the tubing wall, the longitudinal bead including one or more optical fibers disposed therein.

2. The integrated tubing of claim 1, wherein the integrated tubing has one lumen.

3. The integrated tubing of claim 2, wherein the longitudinal bead is incorporated into the tubing wall.

4. The integrated tubing of claim 1, wherein the integrated tubing has two or more lumens.

5. The integrated tubing of claim 4, wherein each lumen of the two or more lumens has approximately a same volume as another lumen of the two or more lumens in an arbitrary length of the integrated tubing.

6. The integrated tubing of claim 4, wherein at least one lumen of the two or more lumens has a different volume than another lumen of the two or more lumens in an arbitrary length of the integrated tubing.

7. The integrated tubing of claim 4, further comprising one or more septa of the integrated tubing, each lumen of the two or more lumens further defined at least in part by the one or more septa.

8. The integrated tubing of claim 7, wherein the longitudinal bead is incorporated into the tubing wall.

9. The integrated tubing of claim 7, wherein the longitudinal bead is incorporated into at least one septum of the one or more septa.

10. The integrated tubing of claim 7, wherein the longitudinal bead is incorporated into each septum of the one or more septa.

11. The integrated tubing of claim 1, wherein the longitudinal bead has one optical fiber disposed therein.

12. The integrated tubing of claim 1, wherein the longitudinal bead has a bundle of optical fibers disposed therein.

13. The integrated tubing claim 1, wherein the integrated tubing is bump tubing.

14. A medical device, comprising:
a catheter tube including:
a catheter-tube wall of the catheter tube; and
one or more lumens of the catheter tube, each lumen of the one or more lumens defined at least in part by the catheter-tube wall; and
an integrated stylet of one or more optical fibers, the catheter tube including a longitudinal bead between opposing sides of the catheter tube having the one or more optical fibers disposed therein.

15. The medical device of claim 14, wherein the catheter tube has one lumen.

16. The medical device of claim 15, wherein the longitudinal bead is incorporated into the catheter-tube wall.

17. The medical device of claim 15, wherein the catheter tube has two or more lumens.

18. The medical device of claim 17, wherein each lumen of the two or more lumens has approximately a same volume as another lumen of the two or more lumens in an arbitrary length of the catheter tube.

19. The medical device of claim 17, wherein at least one lumen of the two or more lumens has a different volume than another lumen of the two or more lumens in an arbitrary length of the catheter tube.

20. The medical device of claim 17, further comprising one or more septa of the catheter tube, each lumen of the two or more lumens further defined at least in part by the one or more septa.

21. The medical device of claim 20, wherein the longitudinal bead is incorporated into the catheter-tube wall.

22. The medical device of claim 20, wherein the longitudinal bead is incorporated into at least one septum of the one or more septa.

23. The medical device of claim 20, wherein the longitudinal bead is incorporated into each septum of the one or more septa.

24. The medical device of claim 14, wherein the longitudinal bead has one optical fiber disposed therein.

25. The medical device of claim 14, wherein the longitudinal bead has a bundle of optical fibers disposed therein.

26. The medical device of claim 14, wherein a distal-end portion of the catheter tube has a smaller diameter than a proximal-end portion of the catheter tube, the distal-end portion of the catheter tube configured to interface with a patient's anatomy and the proximal-end portion of the catheter tube configured to interface with procedural equipment or instrumentation.

27. The medical device of claim 14, wherein the medical device is a peripherally inserted central catheter or a central venous catheter.

28. A method for manufacturing integrated tubing, comprising:
threading at least one optical fiber through a die of an extruder;
forcing a molten polymeric material through the die around the optical fiber to form the integrated tubing with the optical fiber disposed within a longitudinal bead of the integrated tubing; and
cooling the integrated tubing in a cooling bath by pulling the integrated tubing through the cooling bath with a puller.

29. The method of claim 28, further comprising coiling the integrated tubing onto a spool of the integrated tubing with a coiler.

30. The method of claim 28, wherein a rate of pulling the integrated tubing with the puller is increased to create portions of the integrated tubing with a smaller diameter or decreased to create portions of the integrated tubing with a larger diameter.

31. The method of claim 30, further comprising cutting the integrated tubing into predetermined lengths of the integrated tubing with a cutter.

32. The method of claim 31, wherein the cutter is synchronized with the puller such that each predetermined length of the integrated tubing includes a first-end portion of the predetermined length having a smaller diameter than a second-end portion of the predetermined length.

33. The method of claim 32, wherein each predetermined length of the integrated tubing is a catheter tube, the first-end portion of the predetermined length of the integrated tubing being a distal-end portion of the catheter tube configured to interface with a patient's anatomy and the second-end portion of the predetermined length of the integrated tubing being a proximal-end portion of the catheter tube configured to interface with procedural equipment or instrumentation.
